(19) United States
(12) Patent Application Publication
(10) Pub. No.: US 2009/0105664 A1
(43) Pub. Date: Apr. 23, 2009

(54) Syringe Adapter

(76) Inventors: Remedios Dato, Pleasanton, CA (US); Koon-wah Leong, Sunnyvale, CA (US); Steven Neuenfeldt, Pleasanton, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/248,994

(22) Filed: Oct. 10, 2008

Related U.S. Application Data
(60) Provisional application No. 60/981,321, filed on Oct. 19, 2007.

(51) Int. Cl. A61M 5/31

(52) U.S. Cl. 604/207

(57) ABSTRACT

The present invention includes syringes for dispensing liquids. The devices of the invention increase ease of volume control and hence application thereof while minimizing any spillage or fluid migration up the side of the syringe tip.

### FIELD OF THE INVENTION

The invention relates to syringes for dispensing liquids. In particular, a syringe with an adapter useful in dispensing very small amounts of liquid is provided.

### BACKGROUND OF THE INVENTION

In many medical and laboratory applications, it is necessary to provide or administer a single or precisely measured dose of a liquid agent, such as blood, medication or reagent. One such application in which precise amounts of fluid are required is in the patient use of systems for measuring a bodily analyte in a physiological fluid. Such systems typically include test strips containing a reagent material to which a physiological sample is applied. Meters are configured to receive such test strips and determine the analyte concentration of the sample. Prior to the use of such meters and test strips, they are typically checked by methods in which a monitoring agent, often called a control solution is used to test the accuracy and efficacy of the test strips.

Control solutions are often packaged in a plastic or glass container. The solution is then dispensed using a syringe, but it is generally difficult to accurately control the amount of control solution dispensed from syringes. Although advancements are rapidly being made in the development of systems and devices for measuring analyte concentrations, there has been limited advancement in the area of control solution containment and dispensing for use with these advanced systems and devices. Commercially available syringes typically have a tapered dispensing tip that performs reliably on diagnostic test strips where a large volume, e.g. 5 to 20 microliters, of control solution or a body fluid is required. However, such syringes are less accurate at dispensing smaller volumes required by today's more advanced test strips that use less than one microliter.

As the sample volume requirement of some commercially available test strips reaches sub-microliter level, an excess amount of control solution may be delivered by the existing syringes, creating inconvenience and annoyance to the user. For example it can be difficult to aim a large droplet precisely onto the reaction zone of a test strip while also holding the syringe and pushing the plunger.

### BRIEF DESCRIPTION OF THE FIGURES

FIG. 1 is an illustration of an example of a prior art syringe used for containing and dispensing a control solution.

FIG. 2 is a close-up perspective view of the tip of the syringe illustrated in Figure.

FIG. 3 is an illustration of the way in which a control solution may be applied to a strip using a syringe.

FIG. 4a is a side plan cross-sectional view of an embodiment of an adapter according to the present invention for use with a conventional syringe.

FIG. 4b is a top-plan view (along A-A of FIG. 4a) of an embodiment of an adapter according to the present invention for use with a conventional syringe.

FIG. 5a is a side plan cross-sectional view of a further embodiment of an adapter according to the present invention for use with a conventional syringe.

FIGS. 5b is a top-plan view (along A-A of FIG. 5a) of a further embodiment of an adapter according to the present invention for use with a conventional syringe.

FIG. 6 is a side plan cross-sectional view of an embodiment of an adapter according to the present invention.

FIG. 7 is a side plan cross-sectional view of a further embodiment of an adapter according to the present invention.

### DETAILED DESCRIPTION OF THE INVENTION AND PREFERRED EMBODIMENTS

The present invention will be described in the context of analyte concentration measurement applications, and particularly in the context of glucose concentration in blood. However, such is not intended to be limiting and those skilled in the art will appreciate that the subject devices, systems and methods are useful in the measurement of other physical and chemical characteristics, e.g., blood cholesterol level, or other biological substances, e.g., urine, saliva, etc., involving the use of a reagent. Likewise, the present invention may be used in relation to other liquid substances or agents that also require the convenient provision of a precise dose.

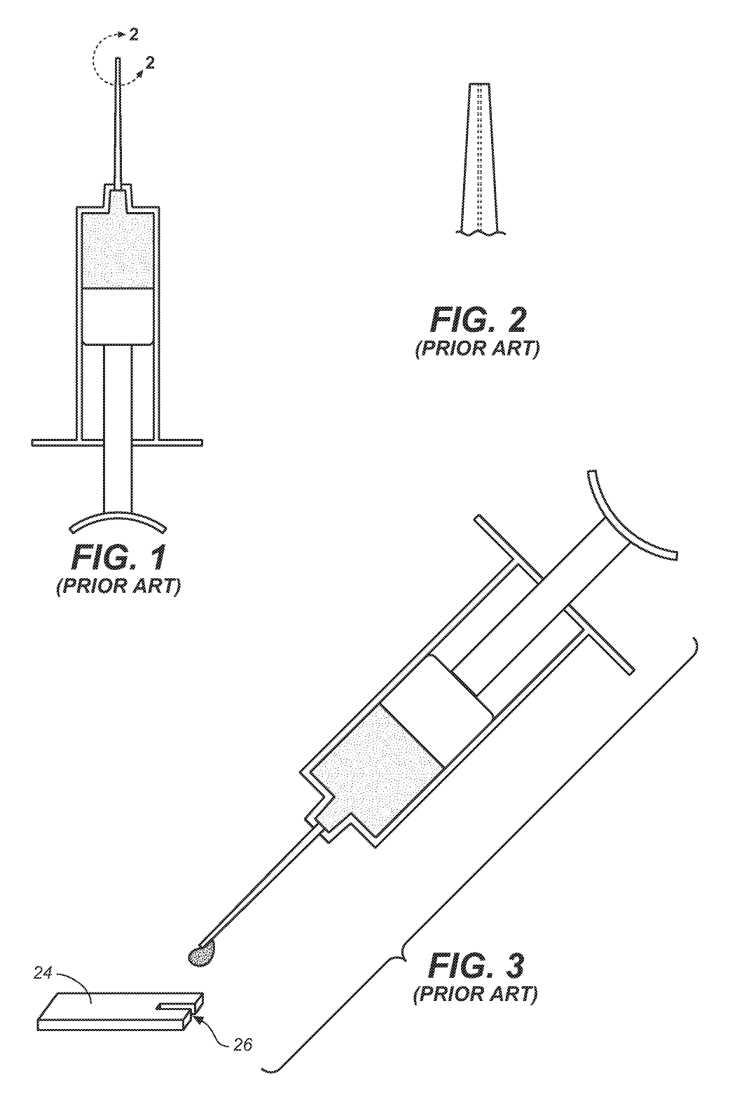

FIG. 1 illustrates an example of a prior art syringe used for containing and dispensing a control solution. FIG. 2 is a close-up perspective view of the tip of the syringe illustrated in FIG. 1. FIG. 3 is a depiction of the syringe of FIG. 1 being used to deliver a fluid to a test strip.

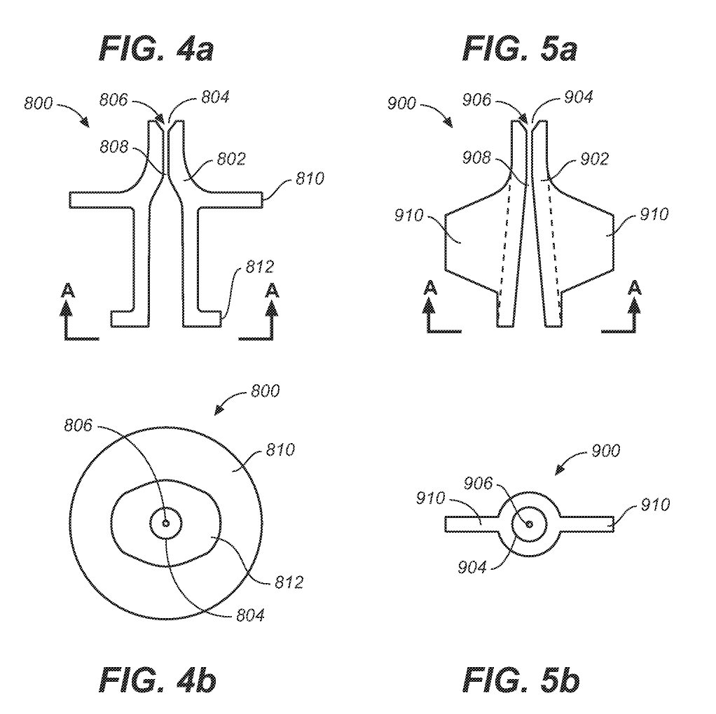

FIG. 4a is a side plan cross-sectional view of an example embodiment of an adapter 800 for use with a conventional syringe (not shown) including a dispensing tip 802, an expanded reservoir 804, a fluid outlet 806, a narrow tapering internal channel 808, a circular disc 810, a means for connection 812 and an arrow ‘A’ depicting the direction of connection of adapter 800 to the tip of a conventional syringe. FIG. 4b is a top plan view of the adapter 800 of FIG. 4a also showing the expanded reservoir 804, the fluid outlet 806, the circular disc 810 and a means for connection 812.

The provision of a reservoir of a fixed volume makes it easier to dispense the required amount of liquid from the syringe because the fluid is held in a secure manner and prevented from falling off the tip. Dispensing the fluid only, as for example, onto a test strip, requires the fluid only to emerge partially from the reservoir so that when the fluid touches a test strip, it automatically migrates to the test strip through capillary action. The volume of the reservoir can be configured so that it is larger than required for testing one test strip. The precise dimensions of the reservoir will depend on the application requirements. For dispensing liquids at or less than the micro-liter amount, the dimension of the reservoir may be about 1-5 mm in diameter and more typically close to 3 mm, and a height in the range of approximately 0.5-1.5 mm, and more typically close to 0.7 mm.

FIG. 5a is a side plan cross-sectional view of a further example embodiment of an adapter 900 for use with a conventional syringe (not shown) including a dispensing tip 902, an expanded reservoir region 904, a fluid outlet 906, a narrow tapering internal channel 908, two flat gripping surfaces 910 located directly opposing each other and an arrow ‘A’ depicting the direction of connection of adapter 900 to the tip of a conventional syringe. FIG. 5b is a top plan view of the adapter 900 of FIG. 5a also showing the expanded reservoir region 904, the fluid outlet 906 and the two flat gripping surfaces 910.

Hospitals use IV and arterial access lines to monitor and infuse medication to patients, especially those in critical or intensive care. For patients who require intensive blood glucose measurements, blood samples are routinely drawn directly from the IV or arterial lines using a small syringe. Blood from the syringe is then applied to a glucose test strip for measurement of the glucose concentration.

As modern glucose monitoring devices make advancements in areas of quick test time and small sample size, a single drop of blood or other liquid delivered from a syringe, typically in the order of 20 to 40 μL, far exceeds the test strip volume requirement. Therefore in many instances, the excess is spilled on work surfaces, flooding the test strip and potentially contaminating the blood glucose-measuring device. A major cause of the spill is the tapered shape of the syringe tip, which is unable to retain the blood.

FIGS. 4 and 5 provide two embodiments of a dispensing tip adapter specifically for use with a conventional syringe, according to the present invention. The adapter is intended to include any or all of the same dispensing features according to the present invention described herein, as shown and described in relation to FIGS. 5 to 15.

The embodiments of FIG. 4 and 5 have some similarities in that they each include an expanded reservoir, a tapering internal channel, a means to ease gripping and handling, and a method of connection to a syringe. The expanded openings or reservoir regions, items 804, 904 located at the tip of the adapters 800 and 900 respectively, facilitate dosing of a small amount of liquid, such as blood, onto the receiving portion of a test strip, for example in the range of 1 to 2 μL or even sub-microliter. It will be apparent to a person skilled in the art that the exact size and shape of reservoirs 804, 904 may vary according to the present invention and are not intended to be restricted to those shown.

The adapter 800 of FIG. 4 includes a circular disc 810 to provide the user with a means of gripping and handling the small component, particularly during the procedure of fitting adapter 800 onto the end of a conventional syringe. Circular disc 810 is significantly wider than the diameter of adapter tip 800 and therefore allows the user to grip it more securely. FIG. 5 shows another embodiment of an adapter tip 900 that includes two, optionally opposing flat gripping surfaces 910 to provide the user with a suitably large surface area to facilitate manipulation of the dispensing tip adapter 900 i.e. particularly during the procedure of fitting the adapter 900 onto the tip of a conventional syringe prior to use.

Attachment of adapters 800, 900 onto a conventional syringe is typically in the direction depicted by arrow ‘A’ in each of FIGS. 4 and 5, and many different methods of connection may be known to those skilled in the art and are therefore intended to be included. The end of the adapter that connects to the syringe in direction ‘A’ may be simply a friction fit as shown by the example of FIG. 4, or by a more secure connection, an example being a Luer lock as shown in FIG. 5. A Luer taper is a well-known standardised system of small-scale plumbing fittings typically used for making leak-proof connections between a male-taper fitting and a cooperating female part on medical and laboratory instruments, including hypodermic syringe tips and needles or stopcocks and needles. Luer taper connections may be a Luer ‘slip’ due to the action of friction as shown in FIG. 4, or optionally a Luer ‘lock’ that more securely joins a tabbed hub on the female fitting which engages threads on the male fitting. Such components may be manufactured either from metal and plastic.

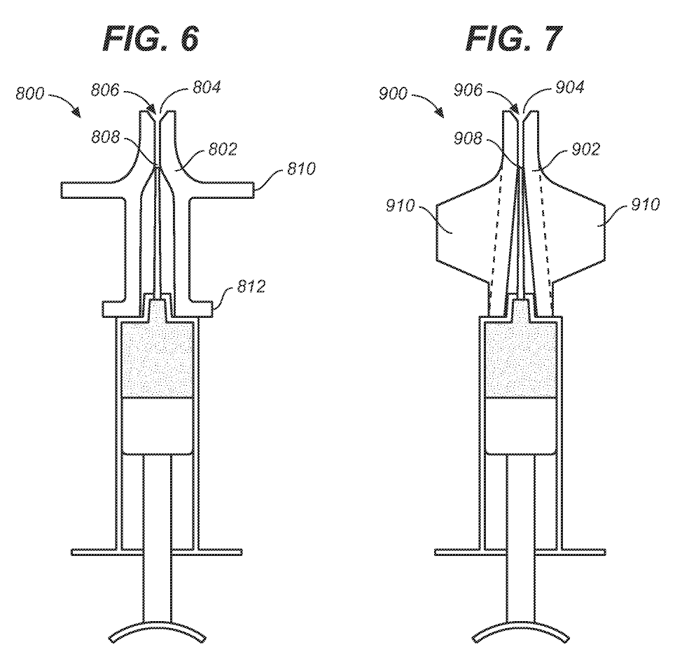

FIG. 6 is a side plan cross-sectional view of an example embodiment of an adapter according to the present invention, including a dispensing tip, attached to a conventional syringe. In FIG. 6, the embodiment of the invention illustrated in FIG. 4 is attached to a conventional syringe such that blood pushed from the end of the syringe needle passes through internal channel 808 and fluid outlet 806 into reservoir 804.

FIG. 7 is a side plan cross-sectional view of a further example embodiment of an adapter according to the present invention, including a dispensing tip, attached to a conventional syringe. In FIG. 7, the embodiment of the invention illustrated in FIG. 5 is attached to a conventional syringe such that blood pushed from the end of the syringe needle passes through internal channel 908 and fluid outlet 906 into reservoir 904.

In one example embodiment the adapter may be made of transparent or minimally translucent polymer in order to provide clear visual indication of test strip dosing. The adapter tip may be fabricated in large quantities using technologies such as injection molding for example. It would be apparent to those skilled in the art that various methods of manufacturing the adapter of the present invention are conceivable and are therefore intended to be included.

There are many advantages provided by the present invention, including the increased ease of sample volume control and hence application thereof giving the user added confidence in the control solution testing procedure. It is also an aim of the dispensing tip of the present invention to minimize any sample spillage and/or fluid migration up the side of the dispensing tip by only providing a volume of control solution that is close to the required volume. Furthermore the volume of fluid is retained at least momentarily localized in the expanded region of the dispensing tip to help ensure adequate sample volume each time a control solution test is performed.

The example embodiments of a fluid-dispensing tip provided herein facilitate the user in aiming the sample onto the sample-receiving region of the test sensor. Improved sample application also virtually eliminates the need to clean up any accidental spills. The fluid-dispensing tip of the present invention also minimizes the dexterity requirement of a user, easing the testing procedure.

What is claimed is:

1. An adapter for use with a conventional syringe, comprising:
a body adapted to fit over the end of the syringe;
an internal channel in the body, wherein a first end of the internal channel is positioned at an outlet of the syringe when the body is positioned on the syringe;
a reservoir at an outlet of the internal channel opposite the inlet wherein the internal channel is wider at the inlet tapering to a narrower opening at the outlet.

2. The adapter of claim 1., wherein the reservoir has a diameter of about 1 to about 5 mm and a height of about 0.5 to about 1.5 mm.
